(19) United States
(12) Patent Application Publication
(10) Pub. No.: US 2006/0252708 A1
(43) Pub. Date: Nov. 9, 2006

(54) Compositions of flavones and long chain fatty acid derivatives isolated from plants and methods related thereto for the control of prostate disorders

(76) Inventors: Xian-Ping Lu, Shenzhen (CN); Song San, Shenzhen (CN); Zhibin Li, Shenzhen (CN); Yanping Luo, Shenzhen (CN); Chenzhong Liao, Shenzhen (CN); Zhiqiang Ning, Shenzhen (CN)

Correspondence Address: FOX ROTHSCHILD LLP;PRINCETON PIKE CORPORATE CENTER, 997 LENOX DRIVE, BUILDING #3, LAWRENCEVILLE, NJ, 08648, US

(21) Appl. No.: 11/124,891

(22) Filed: May 9, 2005

(51) Int. Cl. A61K 31/7024; A61K 31/353

(52) U.S. Cl. 514/27; 514/456

(57) ABSTRACT

Disclosed herein is the extraction, separation, and preparation of plant medicinal extracts to provide compositions containing enriched and isolated flavone derivatives of formula I and long chain fatty acid derivatives of formula II from natural plants, wherein R¹, R², R³, R⁴, R⁵, X, A, B and n₁ are as defined in the specification. These extracts are used to control, i.e., prevent and treat, prostate diseases.

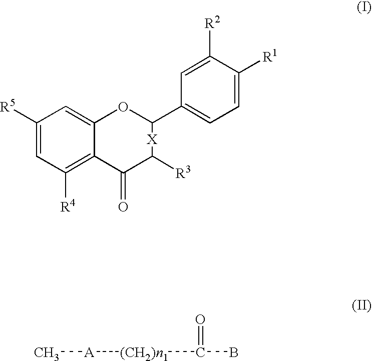

### FIELD OF THE INVENTION

This invention relates to the extraction, separation and preparation of compositions enriched in flavones and long chain fatty acid derivatives derived from plants, and the clinical use of these compositions for controlling prostate disease conditions.

### BACKGROUND OF THE INVENTION

Prostate disease includes prostatitis, benign prostate hyperplasia (BPH), and prostate cancer. Prostate cancer is the most common cancer in the male human population. According to United States data published in 1997, the incidence of prostate cancer ranked number one (41%) among all cancers in males and was the second leading cause of death (14%) in cancer patients. Up to 30% of males over age 50 are found to have prostate cancer upon autopsy. The incidence of prostate cancer in developing countries such as China has increased significantly since 1970s. Thus, prostate cancer is a critical public threat to the worldwide aging population. BPH is the benign form of tissue proliferation in prostate. Although there is no direct evidence shown that BPH leads to prostate cancer, both diseases are believed to be caused by abnormal regulation of hormonal response in male physiology. The normal prostate tissue stops to grow after maturation in man around age 25. The U.S. data has indicated that 50% of men at age 60 and above exhibit BPH. The incidence is further increased to 80% of the male population over 80 years of age. Currently, the major therapeutic method for BPH and prostate cancer is surgical intervention. The method provides a high survival rate for prostate cancer patient diagnosed at the early stage of the disease. However, the survival rate for the middle- and late-stage cancer patients diagnosed is low due to the lack of availability of effective drug treatment.

Prostate specific antigen (PSA) is currently the most important biomarker for early diagnosing prostate cancer. PSA is expressed by the prostate epithelial cells under the induction of androgens and other growth factors such as TGFβ. The normal level of PSA in human serum is in the range of 1-4 ng/ml, but the concentration of PSA in the prostate cancer patients often far over this range; and the concentration of PSA in BPH is also significant higher than that of the normal range, most likely due to the common cause of abnormal hormonal regulation for both prostate cancer and BPH patients in male. Treatments by surgery and drugs for prostate cancer and BHP patients result in decreasing serum level of PSA that often prognoses a better survival rate or relief of the diseases. Therefore, the alteration at PSA level produced by prostate cells can serve as a model system to identify potential effective treatments that have multiple mechanisms of action.

Recent studies shown that the prostate functions not only as a secreting organ participated in the formation of the male ejaculations but also having immune function against invading of bacterial and pathogenic microorganisms by producing immunoglobins and synthesis of zinc-containing anti-bacterial polypeptides. Accordingly, non-surgical drug treatments offer advantages over surgery by preserving the prostate organ for biologcal function. At present, there are four types of drug treatments in clinic use for BPH: (1) antiandrogens, which directly inhibit the action of dihydrotestosterone (DHT); (2) inhibitors of 5α-reductase, which indirectly inhibit the action of DHT; (3) α1 adrenalin receptor blockers, which mainly inhibit contraction and relaxation of smooth muscle; and (4) natural products.

Because the treatments for BPH generally require a long duration of administration, side effects, such as decreased libido, incontinence, and others, often discourage patients from continuing to use the treatments that are otherwise now available. Accordingly, there is an urgent need to now identify effective alternate treatment of prostate pathophysiological disorders with much-improved side effects.

### SUMMARY OF THE INVENTION

The present invention is directed to medicinal compositions which comprise a therapeutically effective amount of at least one isolated flavone derivative of formula I and/or a therapeutically effective amount of at least one isolated long chain fatty acid derivative of formula II 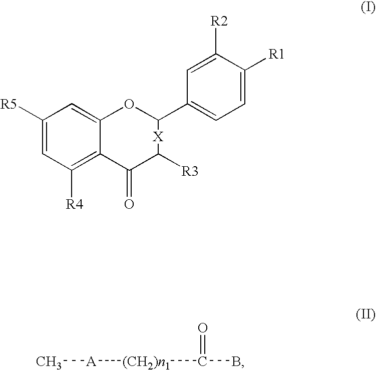
as well as processes for the preparation thereof.

### BRIEF DESCRIPTION OF THE FIGURES

FIG. 1 illustrates the inhibition, by exemplified flavones, of the normal activation the androgen receptor by androgen andrusol

FIG. 2 illustrates the induction of the ER-α receptor by several exemplified flavones.

FIG. 3 illustrates the induction of the ER-β receptor by several exemplified flavones.

FIG. 4 illustrates the inhibition of androgen andrusol conferred by several exemplified long chain fatty acid derivatives.

### DETAILED DESCRIPTION OF THE INVENTION

Mixtures of several Chinese traditional medicinal plants such as flower buds, pollen of rape, and carpet bugle are effective treatment for BPH symptoms, similar to existing anti-androgen or α1-blocker type treatments, however, without the significant side effects. No elemental component ingredients, however, from these example medicinal plants have been identified and/or isolated and characterized with defined pharmacological activity. The term “isolated”, as used herein, refers to compounds separated, from their natural environment. Compositions of the present invention are preferred that are substantially enriched with at least one of the flavone derivatives and/or at least one of the long chain fatty acid derivatives described herein. Compositions of the present invention further encompass substantially purified formulations of the compounds described herein.

The present invention particularly provides plant extracts containing enriched and otherwise isolated flavone derivatives of formula I and long chain fatty acid derivatives of formula II 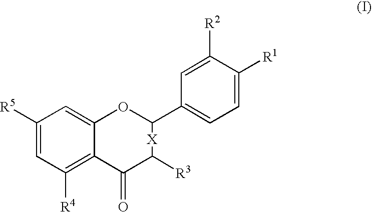
wherein

R¹ is OH, OCH₃, glucosyloxy or rhamnosyloxy;

R² is H, OH or OCH₃;

R³ is H, OH, alkoxy or -Q¹-Q²-(Q³)ₙ, wherein Q¹ is 0, S, or N; Q² is rhamnosyl or glucosyl; Q³ is cinnamyl, cinnamoyloxy containing hydroxyl or benzoyloxy containing hydroxyl, n is an integer from 0 to 5;

R⁴ is OH or alkoxyl;

R⁵ is OH, alkoxyl, glucosyloxy or rhamnosyloxy;

X is a valence bond.

When X is a single bond, R², R³ is H, R¹, R⁴, R⁵ is OH, the compound represented by formula (1) is naringenin;

When X is a double bond, R³ is H, R¹, R², R⁴, R⁵ is OH, the compound represented by formula (1) is luteolin;

When X is a double bond, R² is H, R¹, R³, R⁴, R⁵ is OH, the compound represented by formula (1) is kaempferol;

When X is a double bond, R² is H, R¹, R⁴, R⁵ is OH, R³ is Q¹-Q²-(Q³)ₙ, wherein Q¹ is O, Q² is rhamnosyl, Q³ is cinnamyl, n=1, the compound represented by formula (1) is kaemferol 3-O-(3″-O-cinnamoyl)-α-L-rhamnopyranoside;

When X is a double bond, R² is H, R¹, R⁴, R⁵ is OH, R³ is -Q¹-Q²-(Q³)ₙ, wherein Q¹ is O, Q² is rhamnosyl, Q³ is cinnamyl, n=2, the compound represented by formula (I) is kaemferol 3-O-(2″,3″-O-dicinnamoyl)-α-L-rhamnopyranoside; and, 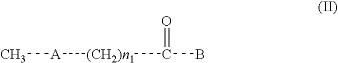
wherein

A is (CH₂)ₙ₂ or (CH₂—CH═CH)ₙ₃, wherein n₂, n₃ are integer from 0 to 20 independently;

B is N-ethoxyl, I-O-fructoside, glyceryl or OH;

n₁ is an integer from 0 to 20;

When A is (CH₂—CH═CH)₃, n₁ is 7, B is glyceryl, the compound represented by formula (II) is linolenic acid glycerin ester;

When A is (CH₂—CH═CH)₃, n₁ is 7, B is N-ethoxyl, the compound represented by formula (II) is N-(2-ethoxyl)-9,12,15-linolenamide;

When A is (CH₂—CH═CH)₃, n₁ is 7, B is 1-O-fructoside, the compound represented by formula (II) is 9,12,15-octadecatrienoic acid 1-O-β-D-fructoside;

When A is (CH₂)₁₃, n₁ is 1, B is 1-O-fructoside, the compound represented by formula (II) is hexadecanoic 1-O-β-D-fructoside.

Naringenin is the aglycone of naringin, which is distributed in the juice and skin of grapefruit, bud of cherry blossom, plum blossom, peach blossom, and pollen of rape. The art is heretofore devoid, however, of any report of naringenin, for example, for the control of prostate disorders.

Luteolin is distributed in flowers and leaves of a great variety of plants, for example, honeysuckle, flos chrysanthemi indici, snow saussurea, selfheal, beautyberry leaf, buhle, and pollen of rape. Moreover, it is also distributed in crusts of the fruits of peanut. The art is heretofore devoid, however, of any report of luteolin, for example, for the control, i.e., prevention and/or treatment, of prostate disease.

Kaempferol is extensively distributed in plants, for example, safflower, ginkgo leaves, impatiens balsamina, crescent euphorbia, kaempferia galamga, and flower pollen of rape. The prior art is heretofore devoid of any report of any kaempferol, for example, for the control, i.e., prevention and/or treatment, of prostate disease. There are reports that coriandrum sativum contains kaemferol 3-O-(3″-O-cinnamoyl)-α-L-rhamnopyranoside, but there is no any report about its pharmacological action and potential use as an isolated active pharmaceutical ingredient for prostate disease. Kaemferol 3-O-(2″,3″-O-dicinnamoyl)-α-L-rhamnopyranoside is an example newly isolated chemical entity identified herein from pollen of rape.

Fatty glyceride is distributed in coffee bean, olive oil and rape oil. Palmitin, linolenic acid glycerin ester, and linoleic acid glycerin ester have the medicinal effects in anti inflammation and anti allergy. Linolenic acid glycerin ester is distributed in the fruits and seeds of akebia stem, as well as in maple tissue. The art is heretofore devoid, however, of any report of these isolated active pharmaceutical ingredients, for the control, i.e., prevention and/or treatment, of prostate disease. 9,12,15-Octadecatrienoic acid 1-O-β-D-fructoside and hexadecanoic 1-O-β-D-fructoside are example newly isolated chemical entities described herein

Processes

An example process for the preparation of plant extracts containing isolated flavone derivatives of formula I and long chain fatty acid derivatives of formula II is described as follows.

### EXAMPLE METHOD ONE

Step 1. Extraction

Raw plants were smashed (mascerated, crushed and/or homogenized), and then extracted by water, methanol, ethanol, acetone, methanol-water, ethanol-water, acetone-water or ethyl acetate. The weight ratio of solvent to plant material is generally preferred to be between about 8:1 to about 15:1. Extracting was repeated 1 to 3 times and the filtrates are collected and evaporated under normal pressure or vacuum to give the crude extracts.

Step 2. Degreasing

The crude extracts obtained in step 1 were dissolved in appropriate amount of water, then washed with one of the solvents such as hexane, cyclohexane, chloroform, petroleum ether or ether.

Step 3. Column Chromatography

The water layer collected in step 2 was evaporated under vacuum and then purified by silica gel column chromatography using petroleum ether: ethyl acetate, cyclohexane: ethyl acetate, hexane: ethyl acetate, petroleum ether: acetone, cyclohexane: acetone, or hexane: acetone as eluant. The elute (between about 8:2 to about 6:4) was collected and concentrated to obtain the plant extracts containing isolated flavone derivatives of formula I and long chain fatty acid derivatives of formula II.

Step 4. Single Compound Separation and Identification

The plant extracts obtained in step 3 were further purified by Sephadex LH20 column chromatography using chloroform: methanol (6:4) as eluant, which resulted in identification of nine single compounds mentioned supra.

### EXAMPLE METHOD TWO

Step 1. Degreasing

Raw plants were smashed, and then extracted by hexane, cyclohexane, chloroform, petroleum ether, or ether, then cooled and filtered.

Step 2. Extraction

The filter cake in step 1 was extracted by water, methanol, ethanol, acetone, methanol-water, ethanol-water, acetone-water or ethyl acetate. The weight ratio of solvent to raw plants is about 8:1˜15:1. Extracting was repeated 1 to 3 times and the filtrates are evaporated under normal pressure or vacuum to give the crude extracts.

Step 3. Column Chromatography

The crude extracts in step 2 were purified by silica gel column chromatography using petroleum ether: ethyl acetate, cyclohexane: ethyl acetate, hexane: ethyl acetate, petroleum ether: acetone, cyclohexane: acetone, or hexane: acetone (about 8:2˜6:4) as eluant. The elute (8:2˜6:4) was collected and concentrated to give the plant extracts containing the isolated flavone derivatives of formula I and long chain fatty acid derivatives of formula II.

Step 4. Single Compound Separation and Identification

The plant extracts obtained in step 3 were further purified by Sephadex LH20 column chromatography using chloroform: methanol (6:4) as eluant, which resulted in identification of nine single compounds mentioned supra.

The term “therapeutically effective amount” as used herein refers to amount isolated flavone derivatives of formula I and long chain fatty acid derivatives of formula II for the control of at least one abnormal prostate condition. Unit dosages of the plant extracts and compositions of the present invention which contain isolated flavone derivatives of formula I and long chain fatty acid derivatives of formula II for the prevention and/or treatment of prostate disease is generally within the range of about 0.01 mg to about 1000 mg. Further preferred ranges of these active ingredients comprised within pharmaceutical or otherwise medicinal compositions of the present invention are from about 0.1 mg to about 5000 mg; about 1 mg to about 2500 mg, about 10 mg to about 500 mg, and about 25 mg to about 250 mg, for example, and a pharmaceutically acceptable carrier.

Pharmaceutical compositions of plant extracts containing enriched or isolated flavone derivatives of formula I and long chain fatty acid derivatives of formula H can be in any forms, such as tablets, capsules, soft capsules, solutions, granules, decoctums, pills, pulvis, suspensions, dispersants, syrups, suppositories, injectable solutions and the like. It can also include excipients. The excipients include adhesive (such as polyvinylpyrrolidone, hydroxy propyl methyl cellulose, etc), disintegrating agent (such as sodium carboxymethyl cellulose, low-substituted hydroxypropyl cellulose, etc), diluent agent (such as amylum, powdered sugar, dextrin, microcrystalline cellulose, mannit, lactose, soybean oil, etc), lubricant (such as magnesium stearate, talcum powder, etc), sweeting agent (such as saccharose, fructose, aspartame, etc), stabilizer (such as sodium carboxymethyl cellulose, cyclodextrin, etc) and antiseptic (such as ethylparaben, sodium benzoate, etc).

The following examples are given as illustrations of the invention. It should be understood, however, that the invention is not limited to the specific details set forth in the examples. All parts and percentages disclosed are by weight unless otherwise specified.

### EXAMPLES

### Example I

3 kg of pollen of rape were smashed and 18 L of chloroform was added. The solution was then heated to reflux for 1 hour. After cooled and filtered, 24 L of ethyl acetate was added to the filter cake, heated to reflux, cooled and filtered. The ethyl acetate extraction was repeated 2 times. The ethyl acetate filtrate was distilled to give the crude extract. The crude extract was purified by silica gel column chromatography eluted by gradient with cyclohexane: acetone (9:16:4). The eluted components (8:2˜6:4) were collected and concetrated to give the plant extracts containing isolated flavone derivatives of formula I and long chain fatty acid derivatives of formula II. Nine compounds have been separated and identified from the plant extract by Sephadex LH-20, which were identified as naringenin, luteolin, kaempferol, kaemferol 3-O-(3″-O-cinnamoyl)-α-L rhamnopyranoside, kaemferol 3-O-(2″,3″-O-dicinnamoyl)-a-L-rhamnopyranoside, linolenic acid glycerin ester, N-(2-ethoxyl)-9,12,15linolenamide, 9,12,15-octadecatrienoic acid 1-O-β-D-fructoside and hexadecanoic 1-O-β-D-fructoside.

### Example II

3 kg of peach blossom were smashed and 18 L of petroleum ether was added. The solution was then heated to reflux for 1 hour. After cooled and filtered, 24 L of 80% ethanol was added to the filter cake, soaked for 48 hours and filtered. The ethanol extraction was repeated 3 times. Then the ethanol filtrate was distilled to give the crude oil. The crude oil was purified by silica gel column chromatography eluted by gradient with cyclohexane: acetone (9:1˜6:4). The eluted compounds (8:2˜6:4) were collected and concetrated to give the plant extracts containing isolated flavone derivatives of formula I and long chain fatty acid derivatives of formula II. Nine compounds have been separated and identified from the plant extract by Sephadex LH-20, which were identified as Naringenin, luteolin, kaempferol, kaemferol 3-O-(3″-O-cinnamoyl)-a-L-rhamnopyranoside, kaemferol 3-O-(2″,3″-O-dicinnamoyl) -α-L-rhamnopyranoside, linolenic acid glycerin ester, N-(2-ethoxyl)-9,12,15-linolenamide, 9,12,15-octadecatrienoic acid 1-O-β-D-fructoside and hexadecanoic 1-O-β-D-fructoside.

### Example III

3 kg of peach leaves was smashed and 30 L of 80% ethanol was added. The solution was then heated to reflux, cooled and filtered. The ethanol extraction was repeated 2 times. The filtrate was collected and distilled to give the crude extract, which was dissolved in 2-fold volume of water and washed with petroleum ether. The water layer was concentrated under vacuum and purified by silica gel column chromatography eluted gradiently with cyclohexane: acetone (9:16:4). The eluted (8:2˜6:4) compounds were collected and concentrated to give the plant extracts containing isolated flavone derivatives of formula I and long chain fatty acid derivatives of formula II. Nine compounds have been separated and identified from the plant extract by Sephadex LH-20, which were identified as Naringenin, luteolin, kaempferol, kaemferol 3-O-(3″-O-cinnamoyl)-a-L-rhamnopyranoside, kaemferol 3-O-(2″,3″-O-dicinnamoyl)-a-L-rhamnopyranoside, linolenic acid glycerin ester, N-(2-ethoxyl)-9,12,15-linolenamide, 9,12,15-octadecatrienoic acid 1-O-β-D-fructoside and hexadecanoic 1-O-β-D-fructoside.

### Example IV

3 kg of carpet bugle was smashed and 30 L of 80% ethanol was added. The solution was then heated to reflux, cooled and filtered. The ethanol extraction was repeated 2 times. The filtrate was collected and distilled to give the crude extract, which was dried and added chloroform, heated to reflux. After cooled and filtered, the filter cake was purified by silica gel column chromatography, eluted by gradient with petroleum ether: ethyl acetate (9:1˜6:4). The eluted compounds (8:2˜6:4) were collected and concetrated to give the plant extracts containing isolated flavone derivatives of formula I and long chain fatty acid derivatives of formula II. Nine compounds have been separated and identified from the plant extract by Sephadex LH-20, which were identified as Naringenin, luteolin, kaempferol, kaemferol 3-O-(3″-O-cinnamoyl)-a-L-rhamnopyranoside, kaemferol 3-O-(2″,3″-O-dicinnamoyl) -α-L-rhamnopyranoside, linolenic acid glycerin ester, N-(2-ethoxyl)-9, 12,15-linolenamide, 9,12,15-octadecatrienoic acid 1-O-β-D-fructoside and hexadecanoic 1-O-β-D-fructoside.

### Example V

Naringenin, kaempferol and luteolin are demonstrated to exhibit the pharmacological activity, for example, of the ability to inhibit the secretion of PSA (Prostate Specific Antigen).

For example, in cultured androgen-dependent prostate cancer cells as shown in

TABLE 1

| | Rate of inhibition (%) | | | | | | | | |
|---|---|---|---|---|---|---|---|---|---|
| | Concentration (ug/ml) | | | | | | | | |
| Name of the drug | 1 | 3 | 6.25 | 10 | 12.5 | 20 | 25 | 40 | 50 |
| naringenin | nd | nd | 78 | nd | 84 | nd | 87 | nd | 88 |
| kaempferol | 53 | 60 | 69 | nd | 65 | nd | 66 | nd | 89 |
| luteolin | 37 | 48 | nd | 57 | nd | 67 | nd | 100 | nd |

Note:
nd = not determined at the concentration.

Long chain amides, Octadecatrienoic acid fructoside and Hexadecanoic fructoside are demonstrated to exhibit the pharmacological activity, for example, of the ability to inhibit the secretion of PSA.

For example, in cultured androgen-dependent prostate cancer cells as shown in

TABLE 2

| | Rate of inhibition (%) | | | | | | |
|---|---|---|---|---|---|---|---|
| | Concentration (ug/ml) | | | | | | |
| Name of drugs | 5 | 10 | 12.5 | 20 | 25 | 50 | 100 |
| Long chain amide | 9.7 | nd | 31 | 52 | 63 | 71 | nd |
| Octadecatrienoic acid | 23 | 40 | 57 | nd | 76 | nd | nd |
| fructoside | | | | | | | |
| Hexadecanoic fructoside | 4.5 | nd | 8.4 | nd | 26 | 42 | 76 |

Note:
nd = not determined at the concentration.

An enzyme-linked immunoadsorbent assay was adopted to determine the level of secreting PSA from LNCaP cells, an androgen-dependent prostate epithelia cancer cells, by the above-mentioned flavones. At first, 100 μl polyclonal Ab at 4 μg/m was added to coat the microplate. They were placed for 12 hours at 4° C., and then washed by PBST and sealed the plate with PBST for 1 hour at 37° C. Supernatants from flavones or mock-treated cells were added into the plate to incubate at water bathed for 2 hours at 37° C. After washing, primary Ab at 200 ng/ml was added and incubated for 1 hour at 37° C. After washing, secondary Ab at 1:8000 was then added and incubated for 1 hour at room temperature. After washing, color reaction substrate was added and incubated for 10 min at dark, then added 2 N sulfuric acid to measure absorption at 492 nm.

### Example VI

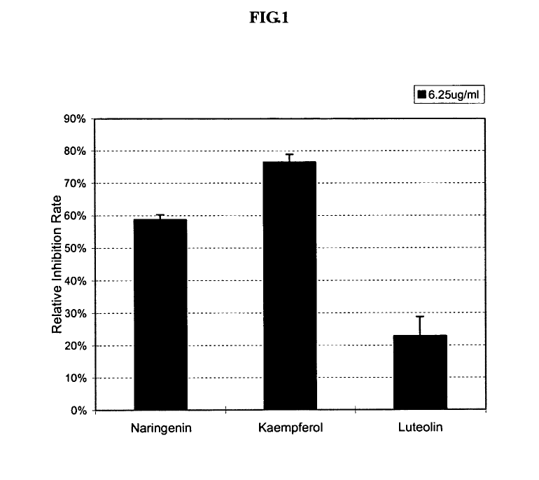

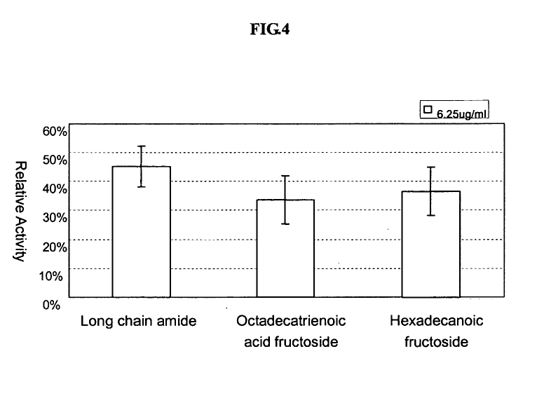

Inhibition of androgen receptor (AR) activation by naringenin, kaempferol, and luteolin and long chain amides, octadecatrienoic acid fructoside, and hexadecanoic fructoside in cells are illustrated in FIG. 1 and FIG. 4, respectively. The activation of AR was performed using a luciferase reporting gene system. The full length AR cDNA was cloned from human's adipose tissue using PCR. The primer sequence 1 used was 5′-cgggatcctggaagattcagccaagctcaagg-3′ (SEQ ID NO:1); the primer sequence 2 used was 5′-gctctagaatgggagggttagatagggaggga-3′ (SEQ ID NO:2). The amplified PCR products were inserted into an expression vector, and then sequenced. The reporter genes were constructed using luciferase detected carrier pGL3-Promoter manufactured by Promega Ltd. Three copies of AR response element were inserted into upstream of pGL3-promoter (element sequence 3 used was 5′-gatctggctctttcagttctaggaagaactgaaagagcctttgggctctttcagttctaggaagaactgaaagagcctttg -3′) (SEQ ID NO:3). The co-transfection experiments were conducted in 96-well plates using U2OS cells. 24 hours after transfection, 10 nM andrusol, DMSO (0.1%), and testing compounds were added into cell culture for further 24 hr incubation. The final cell extracts were prepared and luciferase activities were measured according to manufacture's suggestion.

### Example VII

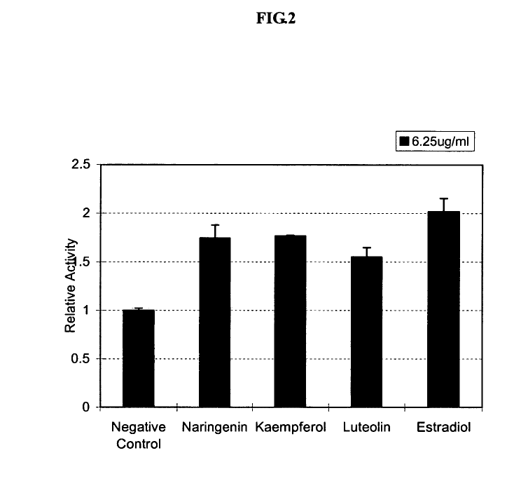

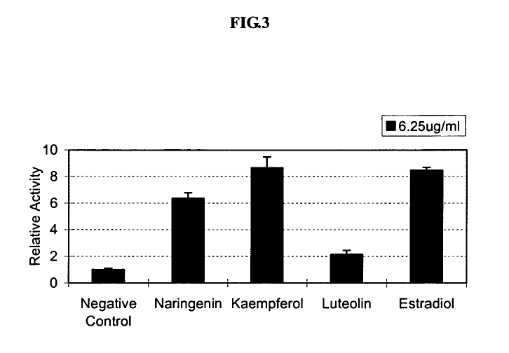

Inhibition of estrogen receptor-α (ERα) and estrogen receptor-β (ERβ) activation by naringenin, kaempferol, and luteolin in vitro are revealed by FIG. 2 and FIG. 3, respectively. The activation evaluation of the ERs were performed using a luciferase reporter gene system. The full length ER-α and ER-β cDNA were separately cloned from human adipose tissue using PCR method. The primer sequence 4 (ERα) used was 5′-ggggtacccctctaacctcgggctgtgct-3′ (SEQ ID NO:4); the primer sequence 5 (ERα) used was 5′-ggaattcgggaatcctcacgcttagtaacata-3′(SEQ ID NO:5); the primer sequence 6 (ERβ) used was 5′-cccaagcttaatgacctttgtgcctcttcttgc-3′ (SEQ ID NO:6); the primer sequence 7 (ERβ) used was 5′-gctctagaggcgtcactgagactgtgggtt-3′(SEQ ID NO:7). The amplified PCR products were inserted into expression vector and confirmed by sequencing. The report genes were constructed by using luciferase detected carrier pGL3-Promoter from Promega Ltd. Three copies of ER response element were inserted into the upstream of pGL3-Promoter (sequence 8 used was 5′-tcgagtcaggtcacagtgacctgatc -3′ (SEQ ID NO:8)). The co-transfection experiments were conducted in 96-well plates using U2OS cells. 24 hours after transfection, 10 nM estradiol, DMSO (0.1%), and testing compounds were added into cell culture for further 24 hr incubation. The final cell extracts were prepared and luciferase activities were measured according to manufacture's suggestion.

What is claimed is:

1. A medicinal composition comprising a therapeutically effective amount of at least one isolated flavone derivative of formula I and/or a therapeutically effective amount of at least one isolated long chain fatty acid derivative of formula II 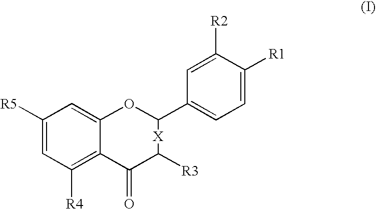
wherein
R¹ is OH, alkoxy, glucosyloxy or rhamnosyloxy;
R² is H, OH or alkoxy;
R³ is H, OH, alkoxy or -Q¹-Q²-(Q³)ₙ, wherein Q¹ is 0, S, or N; Q² is rhamnosyl or glucosyl; Q³ is cinnamyl, cinnamoyloxy containing hydroxyl or benzoyloxy containing hydroxyl, n is an integer from 0 to 5;
R⁴ is OH or alkoxyl;
R⁵ is OH, alkoxyl, glucosyloxy or rhamnosyloxy;
X is a single bond or double bond; and 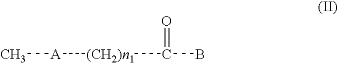
wherein
A is (CH₂)ₙ₂ or (CH₂—CH═CH)ₙ₃, wherein n₂, n₃ are integer from 0 to 20 independently;
B is N-ethoxyl, 1-O-fructoside, glyceryl or OH;
n₁ is an integer from 0 to 20.

2. A medicinal composition according to claim 1 wherein the flavone derivative of formula I:
R¹ is OH;
R² is H;
R³ is H;
R⁴ is OH;
R⁵ is OH;
X is a single bond (naringenin).

3. A medicinal composition according to claim 1 wherein the flavone derivative of formula I:
R¹ is OH;
R² is H;
R³ is OH;
R⁴ is OH;
R⁵ is OH;
X is a single bond (kaempferol).

4. A medicinal composition according to claim 1 wherein the flavone derivative of formula I:
R¹ is OH;
R² is OH;
R³ is H;
R⁴ is OH;
R⁵ is OH;
X is a single bond (luteolin).

5. A medicinal composition according to claim 1 wherein the flavone derivative of formula I:
R² is H;
R³ is -Q¹-Q²-(Q³)ₙ, wherein Q¹ is O; Q² is rhamnosyl; Q³ is cinnamyl, n is 1;
R⁴ is OH;
R⁵ is OH;
X is a double bond (kaemferol 3-O-(3″-O-cinnamoyl)-α-L-rhamnopyranoside).

6. A medicinal composition according to claim 1 wherein the flavone derivative of formula I:
R¹ is OH;
R² is H;
R³ is -Q¹-Q²-(Q¹)ₙ, wherein Q¹ is O; Q² is rhamnosyl; Q³ is cinnamyl, n is 2;
R⁴ is OH;
R⁵ is OH;
X is a double bond (kaemferol 3-O-(2″,3″-O-dicirmamoyl)-α-L-rhamnopyranoside).

7. A medicinal composition according to claim 1 wherein the long chain fatty acid derivative of formula II:
A is (CH₂—CH═CH)ₙ₃, wherein n₃ is 3;
B is glyceryl;
n₁ is 7 (linolenic acid glycerin ester).

8. A medicinal composition according to claim 1 wherein the long chain fatty acid derivative of formula II:
A is (CH₂—CH═CH)ₙ₃, wherein n₃ is 3;
B is N-ethoxyl;
n₁ is 7 (N-(2-ethoxyl)-9,12,15-linolenamide).

9. A medicinal composition according to claim 1 wherein the long chain fatty acid derivative of formula II:
A is (CH₂—CH═CH)ₙ₃, wherein n₃ is 3;
B is 1-O-fructoside;
n₁ is 7 (9,12,15-octadecatrienoic acid 1-O-β-D-fructoside).

10. A medicinal composition according to claim 1 wherein the long chain fatty acid derivative of formula II:
A is (CH₂)ₙ₂, wherein n₂ is 13;
B is 1-O-fructoside;
n₁ is 1 (hexadecanoic 1-O-β-D-fructoside).

11. A medicinal composition according to claim 1 which comprises at least one pharmaceutically acceptable excipient, carrier, or diluent.

12. A medicinal composition according to claim 11 in a dosage form selected from the group consisting of tablet, powder, capsule, solution for oral administration, granule, decoctum, pill, pulvi, suspension, dispersant, syrup, suppository, and solution for parenteral administration.

13. A composition according to claim 11 indicated for the treatment of a prostate pathological disorder or an abnormal prostate condition.

14. The composition of claim 11 in unit dosage form, comprising from 0.01 to 1000 mg of at least one isolated flavone derivative of formula I and/or at least one isolated long chain fatty acid derivative of formula II.

15. The composition of claim 14 in unit dosage form, comprising 0.5 to 500 mg of at least one isolated flavone derivative of formula I and/or at least one isolated long chain fatty acid derivative of formula II.

16. A process for the preparation of a medicinal composition comprising a therapeutically effective amount of at least one isolated flavone derivative of formula I and/or a therapeutically effective amount of at least one isolated long chain fatty acid derivative of formula II 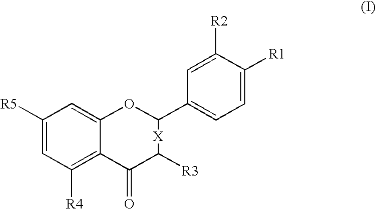
wherein
R¹ is OH, alkoxy, glucosyloxy or rhamnosyloxy;
R² is H, OH or alkoxy;
R³ is H, OH, alkoxy or -Q¹-Q²-(Q³)ₙ, wherein Q¹ is 0, S, or N; Q² is rhamnosyl or glucosyl; Q³ is cinnamyl, cinnamoyloxy containing hydroxyl or benzoyloxy containing hydroxyl, n is an integer from 0 to 5;
R⁴ is OH or alkoxyl;
R⁵ is OH, alkoxyl, glucosyloxy or rhamnosyloxy;
X is a single bond or double bond; and 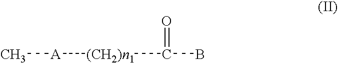
wherein
A is (CH₂)ₙ or (CH₂—CH═CH)ₙ₃, wherein n₂, n₃ are integer from 0 to 20 independently;
B is N-ethoxyl, 1-O-fructoside, glyceryl or OH;
n₁ is an integer from 0 to 20; and
wherein the process comprises the steps of:
smashing plant material,
extracting resulting smashed plant material with at least one solvent, and
purifying the flavone derivatives and/or long chain fatty acid derivatives by column chromatography.

17. The preparation process according to claim 16 wherein an extraction solvent is selected from the group consisting essentially of water, methanol, ethanol, acetone, methanol-water, ethanol-water, acetone-water or ethyl acetate; and,
a method of extracting is reflux extraction or ultrasound extraction.

18. The preparation process according to claim 16, further comprising a degreasing extraction step after the first extracting step wherein a degreasing solvent is selected from the group consisting essentially of hexane, cyclohexane, chloroform, petroleum ether and ether.

19. The preparation process according to claim 16, wherein the chromatography is silica gel column chromatography, polyamine column chromatography, or sephadex LH20 column chromatography.

20. The preparation process according to claim 19, wherein solvents used in gradient elution of silica gel column chromatography comprise two solvents selected from the group consisting essentially of petroleum ether, ethyl acetate, cyclohexane, acetone and hexane.
